(19) United States
(12) Patent Application Publication
(10) Pub. No.: US 2009/0156897 A1
(43) Pub. Date: Jun. 18, 2009

(54) ROTATING SELF-PROPELLED ENDOSCOPE DEVICE

(75) Inventors: Keijiro Omot, Tokyo (JP); Yasuhito Kura, Tokyo (JP); Takahiro Kishi, Kanagawa (JP); Yoshiyuki Tanii, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/066,330

(22) PCT Filed: Sep. 30, 2005
(86) PCT No.: PCT/JP2005/018175

§ 371 Date: Mar. 10, 2008

(51) Int. Cl. A61B 1/00; G08B 23/00

(52) U.S. Cl. 600/118; 340/573.1

(57) ABSTRACT

A rotating self-propelled endoscope device 1 of the present invention comprises an insertion portion 2 to be inserted into a subject, a thrust generation portion 12 provided rotatably around a longitudinal axis of an outer circumference of the insertion portion, a rotary motive force generating unit 3 having a driving unit 45 for rotating the thrust generation portion, a detecting unit 52 for detecting physical information based on driving of the driving unit of the rotation driving portion, and a notifying unit 10 for notifying the physical information based on a detection result of the detecting unit, and a behavior of the thrust generating unit can be grasped by a user.

### TECHNICAL FIELD

The present invention relates to a rotating self-propelled endoscope device that is self-propelled and inserted into a body cavity.

### BACKGROUND ART

As is known, an endoscope is widely used in various fields including medicine and industries with the purpose of observing a portion in a tube or the like that can not be visually checked directly and comprises an elongated insertion portion to be inserted into a portion to be inspected in general.

These endoscopes are known in diversified structures. As one of the examples, a rotating self-propelled endoscope having an insertion portion inserted into a colon per anum is known in which a rotating cylindrical body capable of rotary motion is provided on an outer circumference of the insertion portion around a shaft provided with a helical shape, and by rotating the rotating cylindrical body by a motor or the like, insertion of the insertion portion into the colon can be automatically carried out by a screwing action using friction generated between the helical shaped portion and an intestinal wall.

A technology to insert a medical instrument such as an endoscope into a body cavity using friction between a rotation driving member and a tissue in the body cavity is disclosed in Japanese Patent Application Laid-Open No. 10-113396, for example.

These endoscopes are provided in various types, one of which is a rotating self-propelled endoscope configured to be inserted into a colon per anum in which a rotatable rotating cylindrical body having flexibility is provided on the outer circumference side of the insertion portion around a shaft provided with a helical shaped portion and by rotating the rotating cylindrical body, insertion into the body cavity is automatically carried out. The rotating self-propelled endoscope has a rotation driving portion connected to the insertion portion rotating the rotating cylindrical body around a predetermined shaft.

With the conventional rotating self-propelled endoscope, when the insertion portion is being inserted into the colon, a behavior of the rotating cylindrical body in the body cavity generating thrust by friction with the intestinal wall is not known. Thus, an operator (user) can not grasp nonconformity that rotating speed of the rotating cylindrical body is lowered or idled more than necessary in the colon and the thrust by the screwing action with the intestinal wall is deteriorated. Also, in a bending state in the bending colon, the rotating cylindrical body is preferably rotation-controlled with an optimal rotary torque that can exert a sufficient thrust.

The present invention was made in view of the above circumstances and has an object to provide a rotating self-propelled endoscope with improved insertion performance into a body cavity by grasping a behavior of the rotating cylindrical body in the body cavity from a rotating speed, rotary torque and the like of the rotating cylindrical body of the insertion portion self-propelled and inserted into the body cavity such as a colon.

### DISCLOSURE OF INVENTION

### Means for Solving the Problem

A rotating self-propelled endoscope device of the present invention comprises an insertion portion to be inserted into a subject, a thrust generation portion provided rotatably around a longitudinal axis of an outer circumference of the insertion portion, rotary motive force generating means having driving means for rotating the thrust generation portion, detecting means for detecting physical information based on driving of the driving means of the rotation driving portion, and notifying means for notifying the physical information based on a detection result of the detecting means.

### BRIEF DESCRIPTION OF THE DRAWINGS

FIG. 1 is a view illustrating configuration of a rotating self-propelled endoscope according to a first embodiment of the present invention.

FIG. 2 is a partial sectional view along an insertion axial direction showing configuration of a distal end portion and an insertion portion distal end side of the same.

FIG. 3 is a perspective view illustrating the entire insertion portion of the same.

FIG. 4 is a sectional view illustrating an inside of a rotation driving portion of the same.

FIG. 5 is a block diagram illustrating electrical circuit configuration of the rotating self-propelled endoscope device of the same.

FIG. 6 is a flowchart illustrating an example of an operation to detect a rotating speed and rotary torque of a rotating cylindrical body by the electrical circuit configuration in FIG. 5 of the same.

FIG. 7 is a block diagram illustrating electrical circuit configuration of a rotating self-propelled endoscope device according to a second embodiment.

FIG. 8 is a flowchart illustrating an example of an operation to detect the rotating speed and rotary torque of the rotating cylindrical body by the electrical circuit configuration in FIG. 7 and to store the detected data in a memory device.

FIG. 9 is a block diagram illustrating electrical circuit configuration of a rotating self-propelled endoscope device according to a third embodiment.

FIG. 10 is a flowchart illustrating an example of a control operation by a control circuit to detect the rotating speed and rotary torque of the rotating cylindrical body by the electrical circuit configuration in FIG. 9 of the same.

FIG. 11 is a flowchart illustrating a variation of a control operation by a control circuit by detecting a rotating speed and rotary torque of the rotating cylindrical body by the electric circuit configuration of the variation in FIG. 9.

FIG. 12 is a block diagram illustrating electric circuit configuration of a rotating self-propelled endoscope device according to a fourth embodiment for explaining a magnet for torque detection arranged on one face of a pipe-side pulley.

### BEST MODE FOR CARRYING OUT THE INVENTION

Embodiments of the present invention will be described below referring to the attached drawings.

### First Embodiment

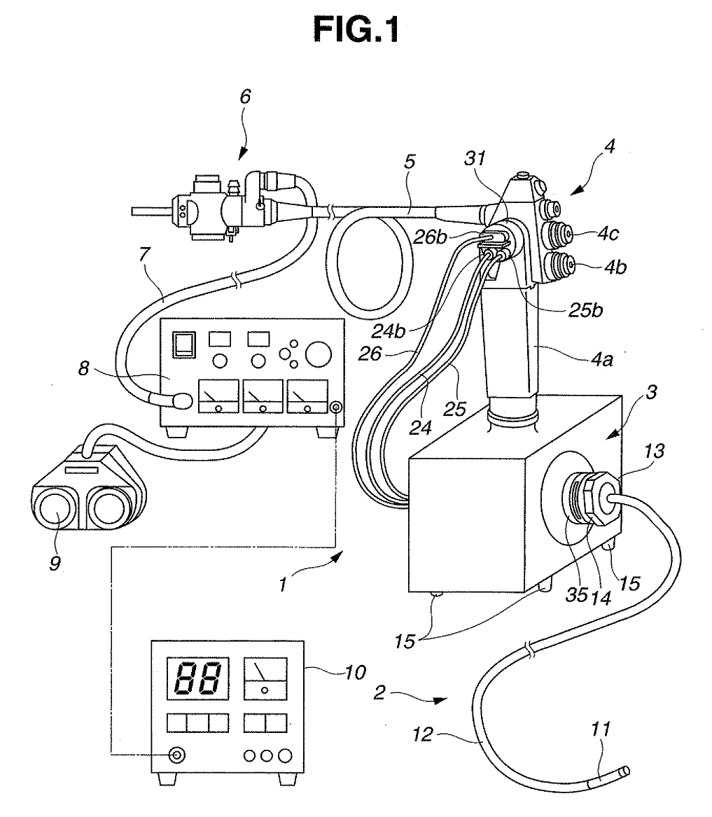

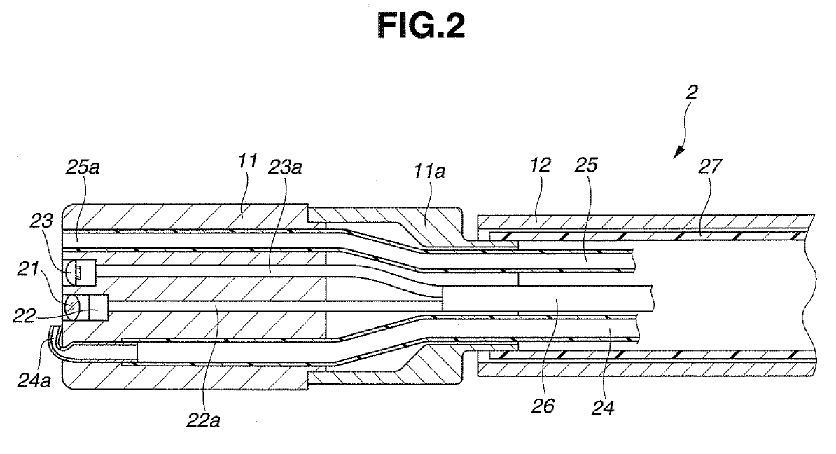

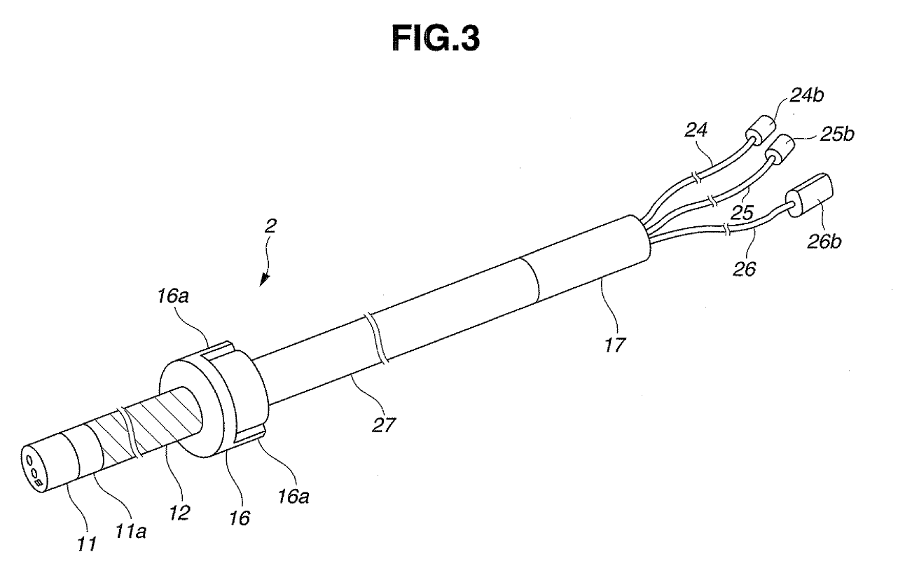

FIGS. 1 to 3 relate to a first embodiment of the present invention, in which FIG. 1 is a view illustrating configuration of a rotating self-propelled endoscope device, FIG. 2 is a partial sectional view illustrating configuration of a distal end portion and an insertion portion distal end side along an insertion axial direction, and FIG. 3 is a perspective view illustrating the entire insertion portion.

As shown in FIG. 1, a rotating self-propelled endoscope device 1 comprises an elongated insertion portion 2 inserted into a body cavity, a rotary motion driving portion 3, which is rotary motive force generating means provided on the proximal end side of the insertion portion 2 and an operation portion 4, a universal cord 5 extended from the operation portion 4, a universal connector 6 provided on the distal end side of the universal cord 5, a control cable 7 extended from the universal connector 6, a control device 8 to which the control cable 7 is detachably connected, for example, a foot switch 9 detachably connected to the control device 8, and a display device 10, which is notifying means detachably connected to the control device 8.

The insertion portion 2 comprises a distal end portion 1 and a rotating cylindrical body 12, which is thrust generating means consecutively provided on the proximal end side of the distal end portion 11. Configuration of the insertion portion 2 provided with the distal end portion 11 will be described in more detail referring to FIG. 2.

As shown in FIG. 2, on a distal end face of the distal end portion 11, an objective optical system 21 is disposed, and an image pickup device 22, which is image pickup means configured by CCD, CMOS and the like, for example, is disposed on an image forming face of the objective optical system 21. Moreover, on the distal end face of the distal end portion 1, an LED 23 to be an illumination light source for illuminating a subject to be a target of shooting by the objective optical system 21 and the image pickup device 22 is provided. A signal line 22a extended from the image pickup device 22 and a signal line 23a, which is a power line extended from the LED 23 are bundled into a signal line in the middle and extended to the proximal end side as a signal cable 26.

On the distal end face of the distal end portion 11, an air/water feeding nozzle 24a is disposed for feeding water for washing the objective optical system 21 and feeding air for wiping water droplets adhering to the objective optical system 21. The air/water feeding nozzle 24a is connected to an air/water feeding tube 24, which is a fluid pipeline, and the air/water feeding tube 24 is extended to the proximal end side.

Moreover, on the distal end face of the distal end portion 11, an opening 25a of a channel 25, which is a fluid pipeline used for suction, for example, is exposed, and the channel 25 is extended to the proximal end side.

Also, on the proximal end side of the distal end portion 11, a hard member to which the distal end side of the rotating cylindrical body 12 is abutted an abutment portion 11a as a thrust receiving portion made of metal, for example, is provided. That is, as will be described later, a distal end portion of the rotating cylindrical body 12 in which a thrust is generated is brought into contact with the abutment portion 11a so that the entire insertion portion 2 including the distal end portion 11 is advanced to the depth direction of the body cavity.

The rotating cylindrical body 12 in the present embodiment is a member in which a metal wire is wound helically and a helical shaped portion to be a helical projection portion (or helical recess portion or a projection portion projected so as to be consecutively provided along the spiral) is formed on its outer circumferential face. In detail, the rotating cylindrical body 12 is a helical tube considering insertion performance into the body cavity and formed to have predetermined flexibility by winding a metal wire made of stainless, for example, and having a predetermined diameter dimension in a single layer. Not limited to the single layer, the metal wire may be wound in multiple threads (two threads, three threads, four threads or the like).

When the metal wire is to be wound helically, close contact between the metal wires can be improved or an angle of the spiral can be set in various ways. In the present embodiment, the rotating cylindrical body 12 in which a helical shaped portion as helical irregularity is formed on the outer circumferential face by winding the metal wire is used as an example, but the rotating cylindrical body may have a helical shape portion in which a helical groove is formed on the outer surface of a tube having flexibility, for example.

The rotating cylindrical body 12 is configured to be capable of rotational movement around an axis in the insertion direction. When the rotating cylindrical body 12 is rotated, the helical shaped portion on the outer circumferential face is brought into contact with an inner wall of the body cavity in a subject, which generates a thrust, and the rotating cylindrical body 12 itself attempts to advance in the insertion direction. At this time, the distal end portion of the rotating cylindrical body 12 is brought into contact with the abutment portion 11a so as to press the distal end portion 11, and a thrust for the entire insertion portion 2 including the distal end portion 11 to advance toward the depth of the body cavity is imparted. The rotating cylindrical body 12 has, as shown in FIG. 3, its proximal end portion connected to a front base 16, which is locking means with a plurality of engagement projection portions 16a formed.

On the inner circumferential face side of the rotating cylindrical body 12, a tube 27 is disposed. The tube 27 has the above-mentioned air/water feeding tube 24, channel 25, and signal cable 26 inserted therethrough for protection so that rotation of the rotating cylindrical body 12 is not prevented on the outer circumferential face side. The tube 27 has its distal end portion connected to the proximal end of the abutment portion 11a, and a fixed pipe 17, which is a hard fixed portion, is connected to the proximal end portion.

The tube 27 has a length in the longitudinal direction longer than the rotating cylindrical body 12, and the air/water feeding tube 24, channel 25, and signal cable 26 are extended from the fixed pipe 17 connected to the proximal end. The air/water feeding tube 24, channel 25, and signal cable 26 inserted through the insertion portion 2 are inserted through the rotary motion driving portion 3 and then, extended to the outside again from the rotary motion driving portion 3 (See FIG. 1).

An air/water connection portion 24b is provided at the end portion of the air/water feeding tube 24, a suction connection portion 25b at the end portion of the channel 25, and a signal connection portion 26b at the end portion of the signal cable 26, respectively, and they are connected to a connection portion 31 (See FIG. 1) provided on the side face of the operation portion 4.

Returning to the explanation of FIG. 1, the insertion portion 2 is connected to a rotary motion transmission portion 14, which is rotary motion transmitting means provided at the rotary motion driving portion 3, and by the connection, a driving force of a motor, which will be described later, provided inside the rotary motion driving portion 3 is transmitted to the rotating cylindrical body 12, by which the rotating cylindrical body 12 is rotated. To the rotary motion transmission portion 14, as will be described later, the insertion portion 2 is detachably attached by screwing with a front retaining member 13.

A grasping portion 4a to be grasped by the hand is provided at the operation portion 4, and various operation buttons such as an air/water feeding button 4b for operating air or water feeding through the air/water feeding tube 24 and a suction button 4c for operating suction through the channel 25 are provided.

In the universal cord 5 extended from the operation portion 4, an air/water feeding pipeline connected to the air/water feeding tube 24, a suction pipeline connected to the channel 25 or a signal line connected to the signal cable 26 are disposed.

The universal connector 6 provided at the distal end side of the universal cord 5 is provided with a connection portion to an air feeding device, a connection portion to a water supply tank, a connection portion to a suction pump, a connection portion to a video processor for processing an image signal from the image pickup device 22 and the like.

In the control cable 7 extended from the universal connector 6, a signal line to the rotary motion driving portion 3 and a signal line to the LED 23 disposed in the distal end portion 11 are disposed.

The control device 8 to which the control cable 7 is connected controls the motor disposed in the rotary motion driving portion 3 or light emitting state of the LED 23 and is provided with a power switch, various volume dials and the like.

The foot switch 9 controls the motor of the rotary motion driving portion 3. However, the foot switch 9 may be used for controlling the light emitting state of the LED 23.

The display device 10 is display means for digitizing and displaying a rotating speed of the rotating cylindrical body 12, a torque, which is a load around a rotating shaft (hereinafter simply referred to as rotary torque), and a driving current value of the motor, which is driving means as will be described later.

In the above-mentioned configuration, the portions other than the insertion portion 2, that is, the rotary motion driving portion 3, operation portion 4, universal cord 5, universal connector 6, control cable 7, control device 8, and foot switch 9 constitute a fluid supply device. Moreover, as the fluid supply device, an air supply device, a water supply tank, a suction pump and the like may be included and a video processor may be further included. Therefore, the rotating self-propelled endoscope device 1 comprises at least a part of the fluid supply device and the insertion portion 2.

On the lower face of the rotary motion driving portion 3, a plurality of leg portions 15 used when mounting the rotary motion driving portion 3 are provided.

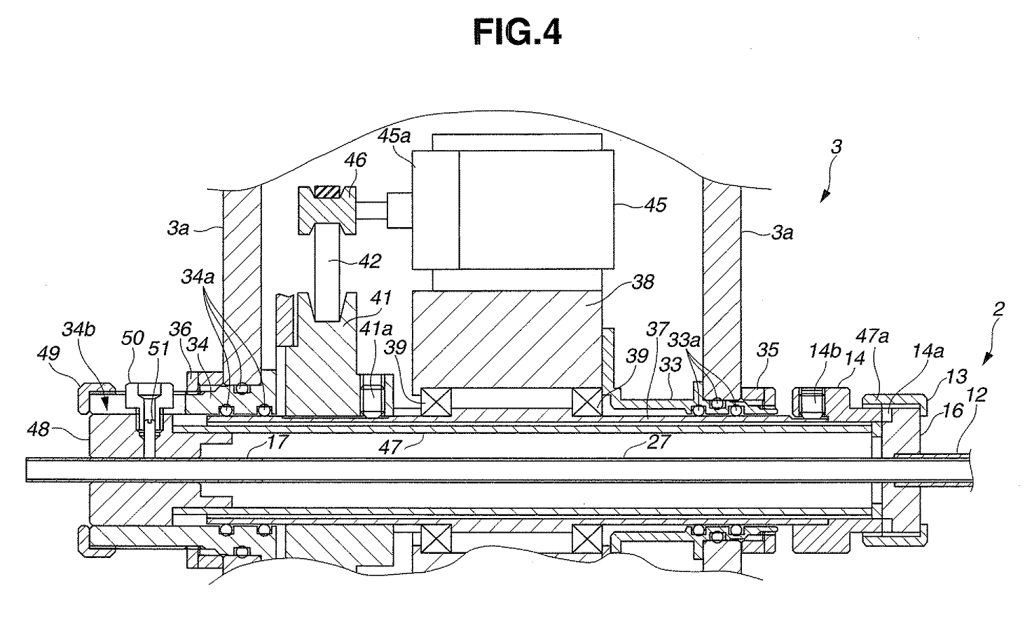

Next, using FIG. 4, internal configuration of the rotary motion driving portion 3 in a state where the proximal end portion of the detachably attached insertion portion 2 is inserted will be described in detail. FIG. 4 is a sectional view illustrating the inside of the rotary motion driving portion 3.

As shown in FIG. 4, the rotary motion driving portion 3 has a case 3a forming an armor. In the case 3a, two hole portions are provided at the front and rear (the direction to which the insertion portion 2 extends is set as the front) so that the insertion portion 2 can be inserted thereto.

At the hole portion on the front side of the case 3a, a substantially cylindrical front holder 33 in which an outward flange is formed in the middle is disposed. The front holder 33 is inserted into the hole portion till the outward flange is brought into contact with the inner face in the vicinity of the hole portion on the front side of the case 3a, and a portion projected forward from the case 3a is fixed to the case 3a by screwing with a front holder retaining ring 35.

At the hole portion on the rear side of the case 3a, a substantially cylindrical rear holder 34 in which an outward flange is formed at one end is disposed. The rear holder 34 is inserted into the hole portion till the outward flange is brought into contact with the inner face in the vicinity of the hole portion on the rear side of the case 3a, and a portion projected rearward from the case 3a is fixed to the case 3a by screwing with a rear holder retaining ring 36.

At each of the holders 33, 34, peripheral grooves are formed one at a portion in contact with the inner circumferential face of each hole portion of the case 3a and two on the inner circumferential face in its vicinity, totaling in three grooves, and O-rings 33a, 34a for waterproof are disposed at each peripheral groove.

In each of the holders 33, 34, a rotating pipe 37 is inserted so as to extend over the holders 33, 34. The rotating pipe 37 is rotated and held by two bearings 39 provided at a frame 38 fixing the front holder 33 and projects forward from an opening portion of the front holder 33.

In the middle of the rotating pipe 37 on the proximal end side (between the bearing 39 and the rear holder 34), a pipe-side pulley 41 is fixed by a fixing screw 41a. The pipe-side pulley 41 is rotated through a pulley belt 42 by rotation of a motor-side pulley 46 of a motor 45 provided with a reducer 45a provided at the frame 38. By the operation, the rotating pipe 37 to which the pipe-side pulley 41 is fixed is rotated with rotation of the pipe-side pulley 41.

The reducer 45a is provided so that a rotating speed of the motor-side pulley 46 by the motor 45 is transmitted and rotates the pipe-side pulley 41 at a desired rotating speed through the pulley belt 42 by a difference in diameter between the motor-side pulley 46 and the pipe-side pulley 41.

In the case 3a of the rotary motion driving portion 3, waterproof against the outside is maintained by each of the O-rings 33a, 34a disposed on the inner circumferential face of each of the holders 33, 34 during the rotation of the rotating pipe 37.

Into the rotating pipe 37, a fixed pipe 47 to which a rear base 48, which is connecting means, is connected at the rear end is inserted. At the rear base 48, a hole into which a fixed pipe 17 connected to a tube 27 of the insertion portion 2 is inserted is formed at a center axis. Also, a plurality of screws 50 (only one of them is shown in FIG. 4) to be projection portions locked by two notch 34b forming a space formed at the rear holder 34 are screwed at the rear base 48 from the outer circumferential direction.

In the screw 50, a hole into which a screw 51 is inserted is formed at the center axis. The screw 51 is screwed with the rear base 48 and presses and fixes the fixed pipe 17 inserted into the rear base 48. Also, at the rear end portion of the rear holder 34, a substantially annular rear retaining member 49 is screwed so as to cover a cut portion of the notch 34b.

Therefore, in the insertion portion 2 passing through each bending portion in a body cavity, by configuring the rear base 48, the fixed pipe 17, and the tube 27 as above, rotation around the axis is regulated and forward and backward movement in the axial direction is easily enabled. That is, the screw 50 screwed to the rear base 48 has its rotation in the direction crossing the axial direction (axial direction connecting the front and the rear of the rotation driving portion 3, that is, in the direction of insertion axis of the insertion portion 2) regulated but becomes capable of moving freely to the front and rear of the rotary motion driving portion 3 in a space formed by the notch 34b of the rear holder 34 and the rear retaining member 49.

By configuration as above, the tube 27 does not follow the rotation of the rotating cylindrical body 12 but its rotation around the axis is regulated. As a result the air/water feeding tube 24, the channel 25 and the signal cable 26 inserted through the tube 27 are prevented from being damaged by twisting.

Also, in the air/water feeding tube 24, the channel 25, and the signal cable 26, generation of a forced load such as pulling and relaxing at forward/backward movement of the tube 27 in the insertion axial direction with respect to the rotating cylindrical body 12 according to the bending state of the insertion portion 2, for example, is prevented.

The rotating pipe 37 has a rotary motion transmission portion 14 fixed to a portion projecting forward by a plurality of screws 14b (only one of them is shown in FIG. 4). By the arrangement, the rotary motion transmission portion 14 is rotated together with the rotating pipe 37. At the rotary motion transmission portion 14, a plurality of engagement grooves 14a (only one of them is shown in FIG. 4), which are engaged means along the axial direction, are formed axially from the end portion on the front side.

To the rotary motion transmission portion 14, the insertion portion 2 is connected by engaging the front base 16 of the insertion portion 2 and screwing the front retaining member 13. At this time, the engagement projection portion 16a, which is engaging means formed at the front base 16 is engaged with the engagement groove 14a of the rotary motion transmission portion 14. By the arrangement, a torque of the rotating pipe 37 is surely transmitted to the insertion portion 2 through the rotary motion transmission portion 14.

In detail, the engagement projection portion 16a of the front base 16 has the side face opposed to its axial direction brought into contact with the side face opposed to the axial direction of the engagement groove 14a of the rotary motion transmission portion 14. Thus, axial rotation of the front base 16 with respect to the rotary motion transmission portion 14 is regulated.

Therefore, the torque of the rotary motion transmission portion 14 is surely transmitted to the front base 16. As a result, the engagement projection portion 16a formed at the front base 16 is engaged with the engagement groove 14a of the rotary motion transmission portion 14 in the rotary motion driving portion 3 so that the torque from the rotating pipe 37 is configured to be surely transmitted to the rotating cylindrical body 12 through the rotary motion transmission portion 14.

Also, the fixed pipe 47 whose rotation is regulated has its distal end portion projected forward to the rotary motion transmission portion 14, and at the distal end face, a sliding ring 47a is disposed. The sliding ring 47a is a member to alleviate friction resistance by contact of the distal end face of the fixed pipe 47 with the proximal end face of the front base 16.

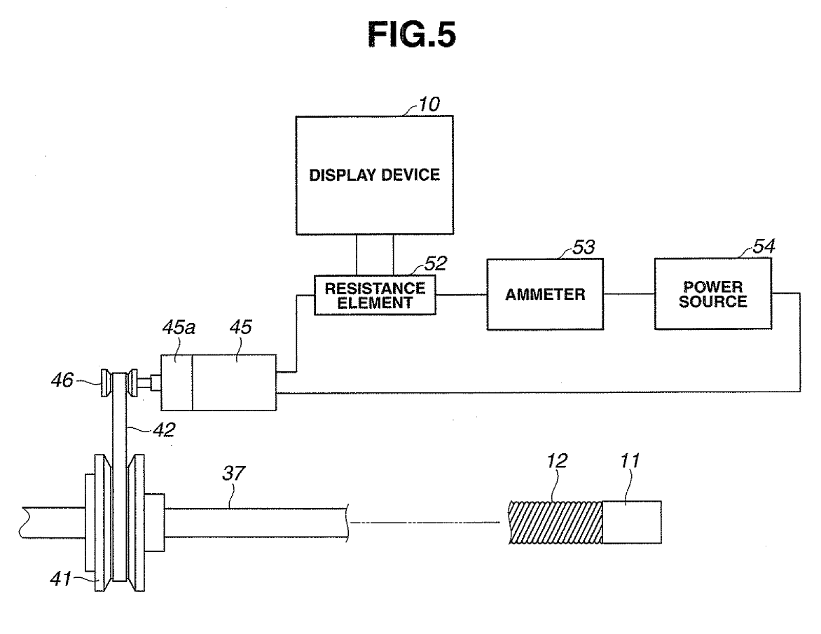

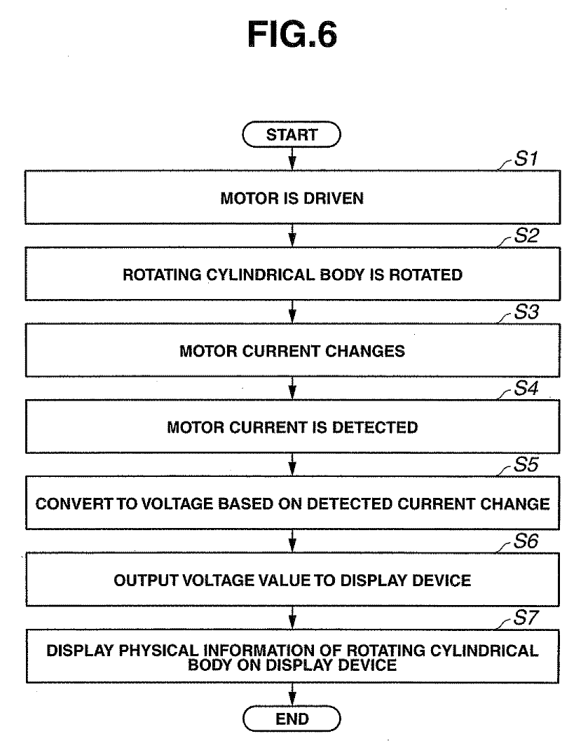

Next, using FIGS. 5 and 6, electrical circuit configuration to detect a behavior of the rotating cylindrical body 12 in the present embodiment by a rotating state of the motor and to notify the state to the display device 10 will be described. FIG. 5 is a block diagram illustrating electrical circuit configuration of the rotating self-propelled endoscope device 1, and FIG. 6 is a flowchart illustrating an example of an operation to detect a rotating speed and rotary torque of the rotating cylindrical body 12 by the electrical circuit configuration in FIG. 5.

As shown in FIG. 5, the display device 10 as external equipment connected to the control device 8 is provided in the rotary motion driving portion 3 and is electrically connected to a resistance element 52, which is detecting means for detecting physical information of the motor 45 through the operation portion 4 and the universal cord 5 shown in FIG. 1. The resistance element 52 is electrically connected in series with the motor 45 and an ammeter 53, and a driving current is supplied from a power source 54 to the resistance element 52, the motor 45, and the ammeter 53.

The resistance element 52 is a resistor that converts the current of the motor 45 to a voltage and outputs the converted voltage to the display device 10 and is a carbon resistor, for example. In the present embodiment, the physical information of the motor 45 uses the resistance element 52 for detecting the rotating speed by an electric current but may use a temperature sensor for detecting a temperature of the motor 45, a vibration sensor for detecting vibration, a noise detection sensor for detecting a noise or the like in order to detect abnormality of the motor 45.

Using a flowchart in FIG. 6, an example of an operation based on each step (S) for detecting the rotary torque of the motor 45 as well as the rotating speed and rotary torque of the rotating cylindrical body 12 of the insertion portion 2 inserted into the body cavity of a subject in the rotating self-propelled endoscope device 1 configured as above will be explained.

First, an operator (user) inserts the insertion portion 2 of the rotating self-propelled endoscope device 1 into a body cavity of a patient from an anus in the case of a colon inspection, for example. And the operator steps on the foot switch 9 so as to switch on and rotate the rotating cylindrical body 12.

At this time, the motor 45 is driven (S1), and the pulley 46 on the motor side is rotated at a predetermined rotating speed and rotary torque by the reducer 45a. And by rotation of the pulley 46 on the motor side, rotation is transmitted to the pulley 41 on the pipe side through the pulley belt 42, and the rotating cylindrical body 12 is rotated at a predetermined rotating speed, rotary torque through the rotating pipe 37, the rotary motion transmission portion 14, and the front base 16 (S2).

And as mentioned above, when the rotating cylindrical body 12 is rotated, the helical shaped portion on the outer circumferential face is brought into contact with an intestinal wall of the subject and a thrust is generated, and the rotating cylindrical body 12 itself is going to travel in the direction of insertion. At this time, the distal end portion of the rotating cylindrical body 12 is brought into contact with the abutment portion 11a and presses the distal end portion 11, and a thrust with which the entire insertion portion 2 including the distal end portion 11 advances toward the depth in the colon is applied.

With an insertion amount of the insertion portion 2 into the colon, friction resistance with the intestinal wall is applied to the rotating cylindrical body 12, which lowers the rotating speed, and a rotary torque for maintaining a predetermined thrust becomes necessary. At this time, a load for maintaining the rotary torque is applied to the motor 45, and a current value of the motor 45 is changed (S3). The ammeter 53 detects a current value of the motor 45 all the time at insertion of the insertion portion 2 into the colon (S4). That is, when the rotary torque of the motor 45 is lowered, the current value for driving the motor 45 is lowered.

The resistance element 52 converts voltage based on the changing current of the motor 45 (S5) and outputs the voltage value on the display device 10 (S6). And the display device 10 digitizes the rotating speed and rotary torque, which is physical information of the rotating cylindrical body 12 based on the detected voltage value inputted from the resistance element 52 and displays it on the display portion (S7).

By the operation, the operator can easily grasp the insertion state of the insertion portion 2 in the bending colon by the rotating speed and rotary torque of the rotating cylindrical body 12 displayed on the display device 10. That is, if the rotating speed in an optimal predetermined range and the rotary torque in a predetermined range set in advance with which the rotating cylindrical body 12 makes a thrust action in contact with the intestinal wall are displayed on the display device 10, insertion of the insertion portion 2 while being propelled without trouble can be grasped.

For example, if the rotating speed or rotary torque in the predetermined range of the rotating cylindrical body 12 is lowered or the rotary torque is increased, nonconformity such that the insertion portion 2 does not generate a sufficient thrust in the colon, the insertion portion 2 receives an excessive torque in the colon or abnormality occurs in the motor 45 in the rotation driving portion 3 is considered to occur. Thus, the operator releases the stepping-on on the foot switch 9 to switch it off and stops the rotation of the rotating cylindrical body 12 once. After that, the operator performs an operation at hand such as twisting of the insertion portion or removal from the colon and rotates the rotating cylindrical body 12 again so as to try insertion of the insertion portion 2 into the colon.

As the result of the above, according to the rotating self-propelled endoscope device 1 in the present embodiment, since the physical information (rotating speed, rotary torque and the like) of the rotating cylindrical body 12 generating a thrust can be obtained from the display device 10 real time in order to grasp the insertion state of the insertion portion 2 inserted into the body cavity, abnormality at insertion of the insertion portion 2 can be easily detected.

A buzzer, an alarm lamp or the like as alarming means that makes an alarm when the rotating speed in the optimal predetermined range and the rotary torque in the predetermined range with which the above rotating cylindrical body 12 makes a thrust action in contact with the intestinal wall become values outside the specified ranges may be provided on the display device 10, or a vibration function as the alarming means may be added to the operation portion 4.

### Second Embodiment

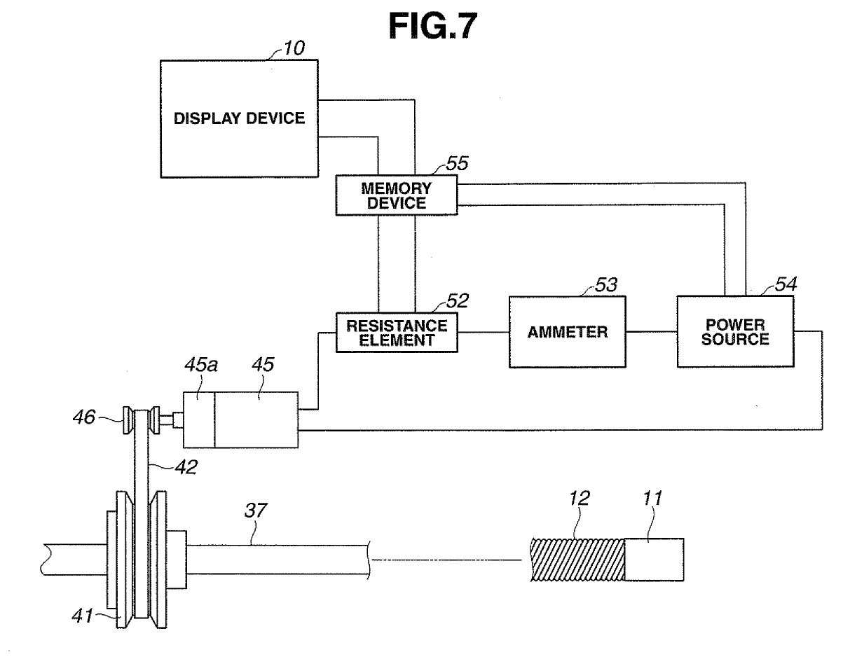

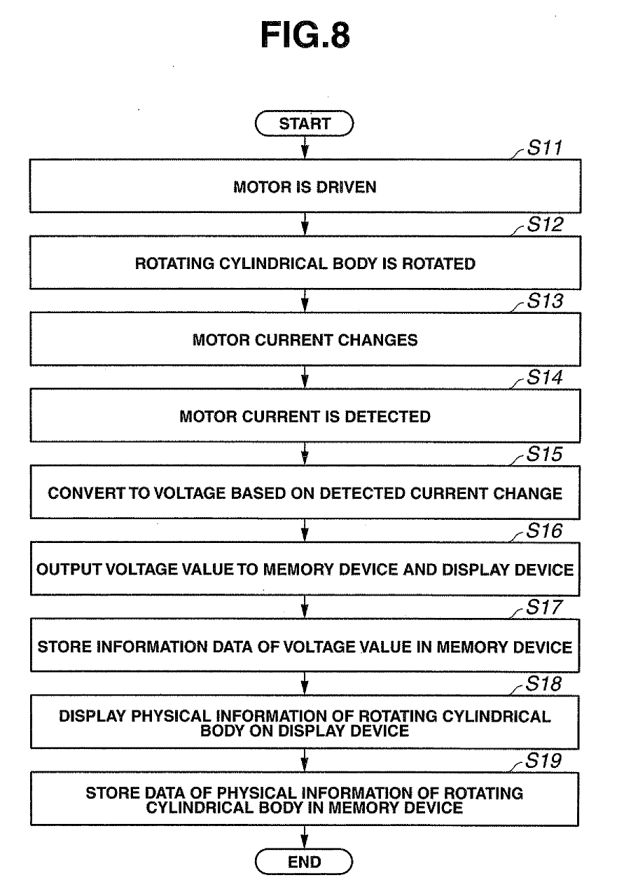

Next, a rotating self-propelled endoscope device according to a second embodiment of the present invention will be described using FIGS. 7 and 8. In the description of the present embodiment, the same reference numerals are used for the same configurations as those in the rotating self-propelled endoscope device 1 in the first embodiment, and the detailed description will be omitted. FIG. 7 is a block diagram illustrating an electric circuit configuration of the rotating self-propelled endoscope device 1 according to the present embodiment, and FIG. 8 is a flowchart illustrating an example of an operation to detect the rotating speed and rotary torque of the rotating cylindrical body 12 by the electric circuit configuration in FIG. 7 and to store the detected data in the memory device.

To the rotating self-propelled endoscope device 1 of the present embodiment, as shown in FIG. 7, a memory device 55, which is a storage medium for storing the rotating speed and rotary torque of the motor 45, is electrically connected to the resistance element 52. The memory device 55 is supplied with power from the power source 54.

Using the flowchart in FIG. 8, an example to detect the rotating speed and rotary torque, which is the physical information of the rotating cylindrical body 12 of the insertion portion 2 inserted into the body cavity (into a colon) of a subject, and to store the detected data in the memory device 55 in the rotating self-propelled endoscope device 1 of the present embodiment configured as above will be described based on each Step (S). Since Step S11 to Step S15 shown in FIG. 8 in the present embodiment are the same as the operation in Step S1 to Step S5 described using FIG. 6 in the first embodiment, the detailed description will be omitted.

In the rotating self-propelled endoscope device 1 of the present embodiment, a voltage value converted by the resistance element 52 at Step S15 is outputted to the display device 10 through the memory device 55 as shown in FIG. 8 (S16). At this time, the memory device 55 stores information data of the voltage value (S17).

The display device 10 into which the voltage value is inputted through the memory device 55 digitizes the rotating speed and rotary torque, which is the physical information of the rotating cylindrical body 12, based on the detected voltage value and displays it on the display portion (S18) and also outputs the physical information of the rotating cylindrical body 12 to the memory device 55.

The memory device 55 stores the inputted data of physical information of the rotating cylindrical body 12 (S19).

As mentioned above, in addition to the advantage of the first embodiment, the rotating self-propelled endoscope device 1 of the present embodiment can store the physical information, which is the voltage value of the motor 45 and an operation history of the rotating cylindrical body 12, by providing the memory device 55 and is configured to be able to utilize the various information as data for repair at a failure or the like.

### Third Embodiment

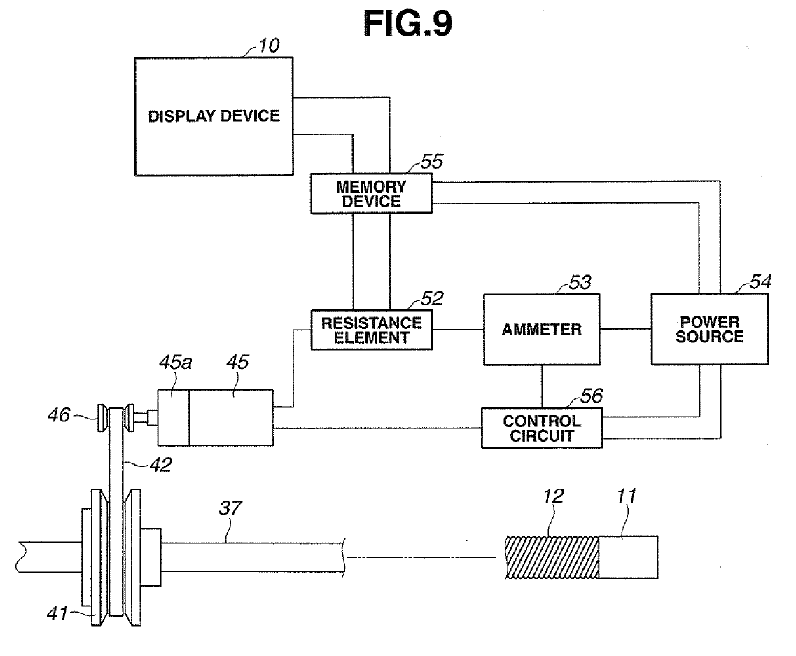

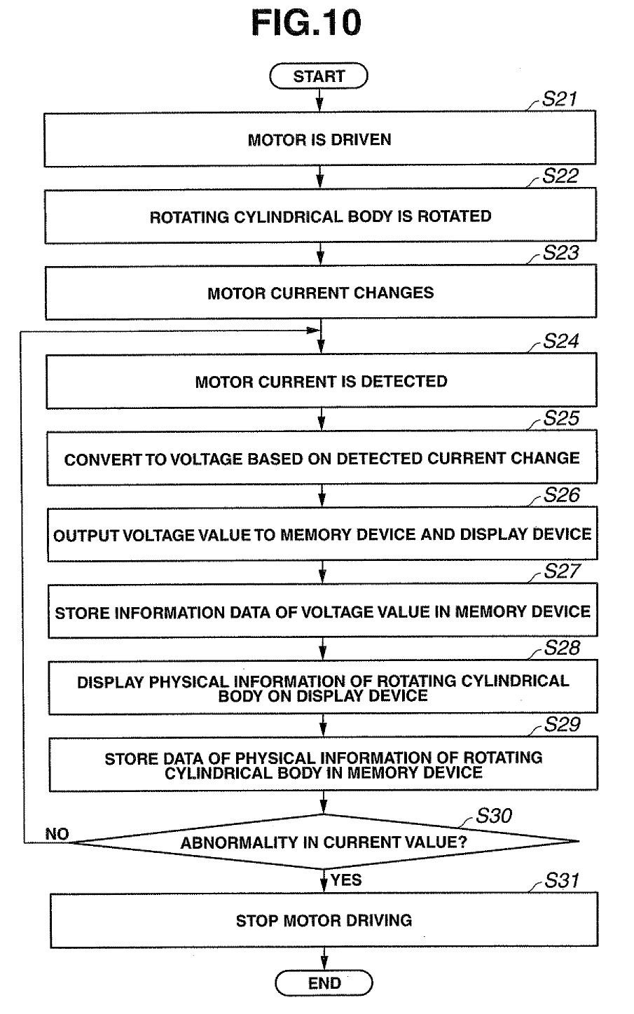

Next, a rotating self-propelled endoscope device according to a third embodiment of the present invention will be described using FIGS. 9 and 10. In the description of the present embodiment, the same reference numerals are used for the same configurations as those in the rotating self-propelled endoscope device 1 in each of the above embodiments, and the detailed description will be omitted. FIG. 9 is a block diagram illustrating electric circuit configuration of the rotating self-propelled endoscope device 1 according to the present embodiment, and FIG. 10 is a flowchart illustrating an example of a control operation by a control circuit to detect the rotating speed and rotary torque of the rotating cylindrical body 12 by the electric circuit configuration in FIG. 9.

In the rotating self-propelled endoscope device 1 of the present embodiment, a control circuit 56 electrically connected in series to the motor 45, the resistance element 52, and the ammeter 53 is provided as shown in FIG. 9. The control circuit 56 is disposed within the rotation driving portion 3, though not shown in FIG. 9, and electrically connected also to the power source 54. A driving current from the power source 54 is supplied to the motor 45, the resistance element 52, and the ammeter 53 through the control circuit 56.

An example of control to detect the rotating speed and rotary torque, which is the physical information of the rotating cylindrical body 12 of the insertion portion 2 inserted into the body cavity (into a colon) of a subject, in the rotating self-propelled endoscope device 1 of the present embodiment configured as above will be described using the flowchart of FIG. 10. Since Step S21 to Step S25 shown in FIG. 10 in the present embodiment are the same as the operation in Steps S1 to S5 described using FIG. 6 in the first embodiment and Step S26 to Step S29 shown in FIG. 10 are the same as the operation in Step S16 to S19 described using FIG. 8 in the second embodiment, the detailed description will be omitted.

In the rotating self-propelled endoscope device 1 of the present embodiment, the control circuit 56 connected to the ammeter 53 monitors a current value supplied to the motor 45, and as shown in FIG. 10, determination on whether the current value is outside a threshold value in a predetermined range, an abnormal current value, or not is made at Step S30 by the control circuit 56 (S30).

At Step S30, if the current value supplied to the motor 45 is within a threshold value in a predetermined range under the determination by the control circuit 56, the routine returns to Step S24 again, while if the current value is outside the threshold value in the predetermined range, the control circuit 56 stops current supply to the motor 45 and stops driving of the motor 45 (S31). The determination made by the control circuit 56 at Step S30 may be made by setting predetermined threshold values for the rotating speed and rotary torque in appropriate predetermined ranges of the rotating cylindrical body 12 and by comparing the rotating speed and rotary torque derived from the current values supplied to the motor 45 with the threshold values in the predetermined ranges.

As mentioned above, the rotating self-propelled endoscope device 1 of the present embodiment can determine abnormality and automatically stop driving of the motor 45 and rotation of the rotating cylindrical body 12 by providing the control circuit 56, in addition to the advantage of the second embodiment, in the case of abnormality of the motor 45 driving the rotating cylindrical body 12 or if the appropriate predetermined rotating speed and rotary torque of the rotating cylindrical body 12 are exceeded.

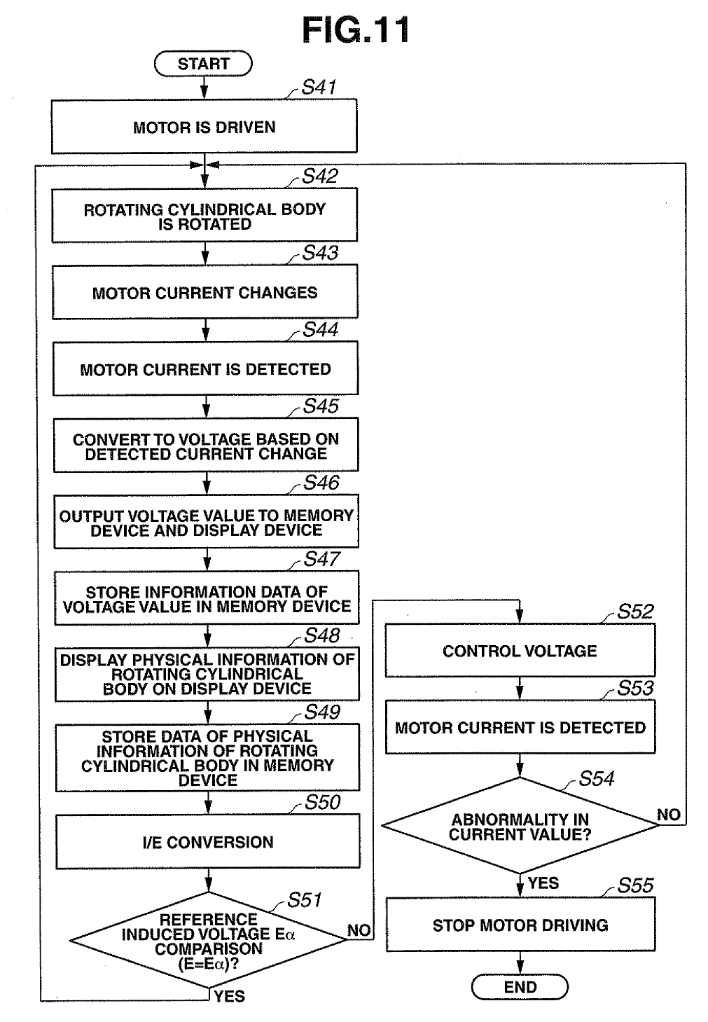

The control circuit 56 may carry out control based on a flowchart shown in FIG. 11. FIG. 11 is a flowchart illustrating a variation of a control operation by the control circuit to detect the rotating speed and rotary torque of the rotating cylindrical body 12 by the electrical circuit configuration in FIG. 9. Since Step S41 to Step S49 shown in FIG. 11 are the same operations as Step S21 to Step S29 described using FIG. 10, the detailed description will be omitted.

After the data of physical information of the rotating cylindrical body 12 is stored in the memory device 55 at Step S49, the control circuit 56 carries out induction voltage E-conversion of the motor 45 based on the current value detected by the ammeter 53 (S50). The control circuit 56 determines if a predetermined reference induction voltage Eα in the motor 45 set in advance and the induction voltage E converted at Step S50 are the same voltage value (E=Eα) or not (S51).

If the control circuit 56 determines that the reference induction voltage Ea in the motor 45 and the induction voltage E are the same voltage value (E=Ea), the routine goes to Step S44 and the routines of Steps S44 to S51 is looped in the insertion process of the insertion portion 2 into the colon. On the other hand, if the control circuit 56 determines that the reference induction voltage Eα in the motor 45 and the induction voltage E are different voltage values (E>Eα, E<Eα), the control circuit 56 changes a supply voltage to the motor 45 so that the reference induction voltage Eα in the motor 45 and the induction voltage E become the same voltage value (E=Eα) (S52).

Next, a current value of the motor 45 is detected by the ammeter 53 (S53), and determination on whether the current value is outside a threshold value in a predetermined range, an abnormal current value, here, or not is made by the control circuit 56 (S54).

At the Step S54, under the determination by the control circuit 56, if the current value supplied to the motor 45 is within a threshold value in a predetermined range, the routine returns to Step S45 again, while if the current value is outside the threshold value in the predetermined range, the control circuit 56 stops current supply to the motor 45 and stops driving of the motor 45 (S55).

As mentioned above, since the rotating self-propelled endoscope device 1 of the present variation can maintain the rotating speed and rotary torque of the motor 45 driving the rotating cylindrical body 12, the rotating cylindrical body 12 acts on the intestinal wall while the predetermined rotating speed and rotary torque set in advance are maintained constant, and insertion performance of the insertion portion 2 into the bending colon is improved in addition to each of the above-mentioned advantages.

### Fourth Embodiment

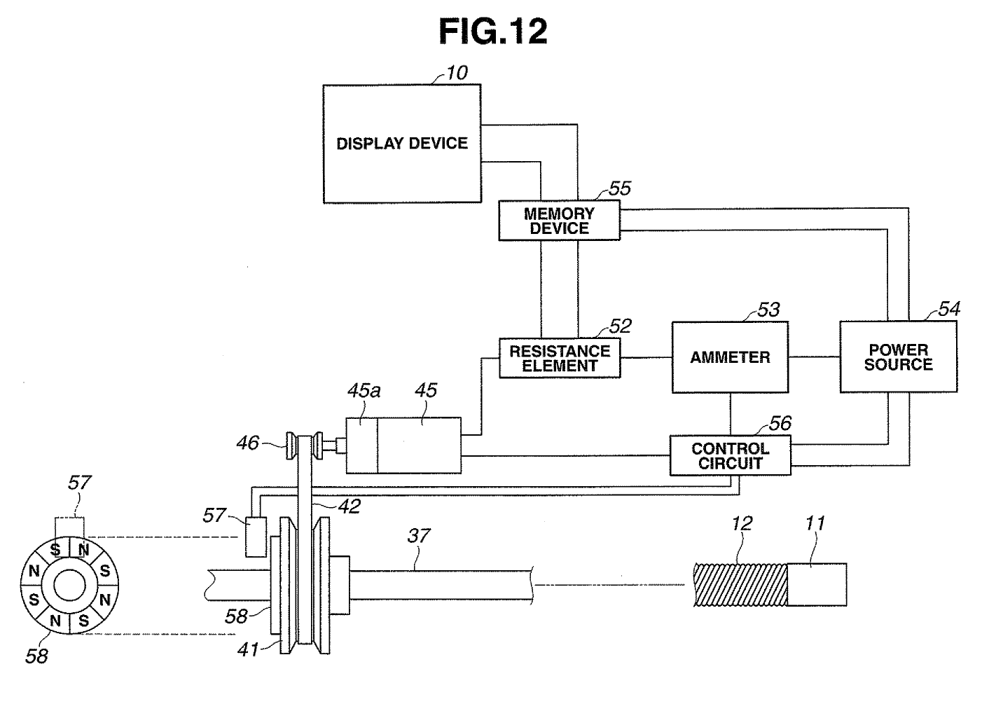

Next, a rotating self-propelled endoscope device according to a fourth embodiment will be described using FIG. 12. In the description of the present embodiment the same reference numerals are used for the same configurations as those in the rotating self-propelled endoscope device 1 in each of the above embodiments, and the detailed description will be omitted. FIG. 12 is a block diagram illustrating electric circuit configuration of the rotating self-propelled endoscope device 1 according to the present embodiment for explaining a magnet for torque detection disposed on one face of the pulley 41 on the pipe side.

The rotating self-propelled endoscope device 1 of the present invention has, as shown in FIG. 12, a magnet 58 for torque detection in which a magnetic body with a plurality of S-poles and a plurality of N-poles arranged alternately in the circumferential direction is disposed on one face of the pulley 41 on the pipe side, on the proximal end face in this case, and a magnetic detection portion 57, which is detecting means for detecting magnetism of the magnet 58 for torque detection.

The magnetic detection portion 57 is electrically connected to the control circuit 56, and the detected magnetism of the S-pole or N-pole of the magnet 58 for torque detection is outputted to the control circuit 56. That is, the magnetic detection portion 57 detects the rotating speed and rotary torque, which is the physical information of the pulley 41 on the pipe side, by passage of the S-pole or N-pole of the magnet 58 for torque detection by rotation of the pulley 41 on the pipe side and outputs the detection result to the control circuit 56.

As a result, the magnetic detection portion 57 can detect the physical information (rotating speed and rotary torque) of the rotating cylindrical body 12 to which the rotating speed and rotary torque of the pulley 41 on the pipe side is transmitted. By the operation, a value detected by the magnetic detection portion 57 can be made into the physical information data of the rotating cylindrical body 12 stored in the memory device 55 described in the third embodiment and into the physical information of the rotating cylindrical body 12 displayed on the display device 10. Moreover, the control circuit 56 can make determination by comparing the rotating speed and rotary torque set in the predetermined range of the rotating cylindrical body 12 based on the value detected by the magnetic detection portion 57.

The threshold value of the physical information data of the rotating cylindrical body 12 can be configured as variable by replacing the resistance element 52 in each of the above embodiments by a potentiometer.

The present invention is not limited to the above embodiments but it is needless to say that various variations and applications are possible in a range not departing from the gist of the invention.

1. A rotating self-propelled endoscope device comprising:
an insertion portion to be inserted into a subject;
a trust generation portion provided rotatably around a longitudinal axis of an outer circumference of the insertion portion;
rotary motive force generating means having driving means for rotating the thrust generation portion;
detecting means for detecting physical information based on driving of the driving means of the rotation driving portion; and
notifying means for notifying the physical information based on a detection result of the detecting means.

2. A rotating self-propelled endoscope device comprising:
an insertion portion to be inserted into a subject;
a thrust generation portion provided rotatably around a longitudinal axis of an outer circumference of the insertion portion;
rotary motive force generating means having driving means for rotating the thrust generation portion;
detecting means for detecting physical information based on driving of the driving means of the rotation driving portion; and
a control portion for controlling rotation of the thrust generation portion based on a detection result of the detecting means.

3. The rotating self-propelled endoscope device according to claim 1, wherein the physical information is a torque amount of the driving means.

4. The rotating self-propelled endoscope device according to claim 1, wherein the physical information is a driving current value of the driving means.

5. The rotating self-propelled endoscope device according to claim 1, wherein the physical information is a torque amount of the thrust generation portion.

6. The rotating self-propelled endoscope device according to claim 1, wherein the notifying means digitizes and displays the physical information.

7. The rotating self-propelled endoscope device according to claim 1, further comprising alarming means for notifying abnormality by an alarm sound, vibration or lighting of a light emitting body when a value of the physical information becomes a value outside a predetermined range.

8. The rotating self-propelled endoscope device according to claim 1, further comprising a storage medium for storing the physical information.

9. The rotating self-propelled endoscope device according to claim 1, wherein the detecting means is a potentiometer which can vary a threshold value of the physical information.

10. The rotating self-propelled endoscope device according to claim 2, wherein the physical information is a torque amount of the driving means.

11. The rotating self-propelled endoscope device according to claim 2, wherein the physical information is a driving current value of the driving means.

12. The rotating self-propelled endoscope device according to claim 2, wherein the physical information is a torque amount of the thrust generation portion.

13. The rotating self-propelled endoscope device according to claim 2, wherein the notifying means digitizes and displays the physical information.

14. The rotating self-propelled endoscope device according to claim 2, further comprising alarming means for notifying abnormality by an alarm sound, vibration or lighting of a light emitting body when a value of the physical information becomes a value outside a predetermined range.

15. The rotating self-propelled endoscope device according to claim 2, further comprising a storage medium for storing the physical information.

16. The rotating self-propelled endoscope device according to claim 2, wherein the detecting means is a potentiometer which can vary a threshold value of the physical information.
